(19) United States
(12) Patent Application Publication
(10) Pub. No.: US 2026/0060837 A1
(43) Pub. Date: Mar. 5, 2026

(54) ANTI-SNORING MOUTHPIECE

(71) Applicant: Yaonan LIANG, Guangzhou (CN)

(72) Inventor: Yaonan LIANG, Guangzhou (CN)

(21) Appl. No.: 18/820,343

(22) Filed: Aug. 30, 2024

(51) Int. Cl. A61F 5/56

(52) U.S. Cl. CPC A61F 5/566

(57) ABSTRACT

An anti-snoring mouthpiece includes an upper mouthpiece and a lower mouthpiece. The upper mouthpiece includes an upper mouthpiece body and an upper dental pad, where the upper mouthpiece body includes a base plate, a first inner baffle, and a first outer baffle. A first accommodating cavity is formed by connecting the base plate, the first inner baffle and the first outer baffle, and the upper dental pad is embedded in the first accommodating cavity. The lower mouthpiece includes a lower mouthpiece body and a lower dental pad, where the lower mouthpiece body includes a top plate, a second inner baffle and a second outer baffle. A second accommodating cavity is formed by connecting the top plate, the second inner baffle and the second outer baffle, and the lower dental pad is embedded in the second accommodating cavity. A through hole is adjusted to adjust a relative position.

### TECHNICAL FIELD

The present disclosure belongs to the technical field of mouthpieces and relates to an anti-snoring mouthpiece.

### BACKGROUND

Snoring is a sleep phenomenon mainly common among obese people. The main reason is that when part of soft tissues such as the soft palate and tongue root subside under the action of gravity, the respiratory tract becomes narrower, such that inhaled air has to forcibly pass through the narrowed space. In this case, air resistance increases, and mucous membranes or secretions in the narrowed portion will vibrate or produce friction sounds. The phenomenon of producing vibration or friction sounds is called snoring. Snoring is a serious threat to health, because snoring causes repeated pauses in breathing and sleep, severe depletion of oxygen in brains and blood, and hypoxemia, and even induces hypertension, cardiovascular and cerebrovascular diseases, arrhythmia, myocardial infarction, angina pectoris, and the like. Respiratory pauses for more than 120 seconds at night easily lead to sudden death in the early morning.

Various anti-snoring mouthpieces have emerged on the market. The anti-snoring mouthpieces orally used are shaped like mouth guards for athletes, and worn in mouths of patients when sleeping. The working principle lies in that the mouthpiece pulls a patient's lower jaw, soft palate and tongue root forward, increases muscle tension of the throat, tongue and soft palate, and opens up the respiratory tract that collapses due to muscle relaxation during sleep, thereby increasing a volume of the airflow passage for the patient to breathe during sleep.

Most of anti-snoring mouthpieces in the prior art are made of soft materials, and are prone to deformation during use, thereby resulting in insignificant anti-snoring effects. Further, after the upper mouthpiece body and the lower mouthpiece body are fixed, the relative position between the upper mouthpiece body and the lower mouthpiece body cannot be adjusted back and forth according to the actual needs of the patient, such that the versatility of anti-snoring mouthpieces in the prior art is poor.

### SUMMARY

Aiming at the defects of the prior art, an objective of the present disclosure is to provide an anti-snoring mouthpiece. The anti-snoring mouthpiece has a simple structure, strong stability and less deformation, and a relative position between an upper mouthpiece body and a lower mouthpiece body is adjustable.

In order to achieve the above objective, the present disclosure adopts a technical solution as follows:

An anti-snoring mouthpiece, including an upper mouthpiece and a lower mouthpiece;
the upper mouthpiece includes an upper mouthpiece body and an upper dental pad, where the upper mouthpiece body includes a base plate, a first inner baffle, and a first outer baffle; a first accommodating cavity is formed by connecting the base plate, the first inner baffle and the first outer baffle, and the upper dental pad is embedded in the first accommodating cavity;
the lower mouthpiece includes a lower mouthpiece body and a lower dental pad, where the lower mouthpiece body includes a top plate, a second inner baffle and a second outer baffle; a second accommodating cavity is formed by connecting the top plate, the second inner baffle and the second outer baffle, and the lower dental pad is embedded in the second accommodating cavity;
the upper mouthpiece body and the lower mouthpiece body are made of hard plastic, and the upper dental pad and the lower dental pad are made of flexible rubber; circumferences of the upper mouthpiece body and the lower mouthpiece body are smaller than those of the upper dental pad and the lower dental pad respectively, and the upper dental pad and the lower dental pad extend from both ends of the upper mouthpiece body and the lower mouthpiece body and extend backward respectively;
a bottom end of the base plate of the upper mouthpiece body is provided with an airway protrusion, and when the airway protrusion abuts against a top end of the top plate of the lower mouthpiece body, an airway for airflow to pass through is formed between the upper mouthpiece body and the lower mouthpiece body; and
the top end of the top plate of the lower mouthpiece body is provided with a limiting boss, a plurality of through holes are formed in the limiting boss, the bottom end of the base plate of the upper mouthpiece body is provided with a limiting insertion strip, the limiting insertion strip is located in the through hole to fix the upper mouthpiece and the lower mouthpiece, and the through hole corresponding to the limiting insertion strip is adjusted to adjust a relative position between the upper mouthpiece and the lower mouthpiece.

Further, a notch is formed in a middle of the first inner baffle of the upper mouthpiece body.

Further, the airway protrusion is located at an outer edge of a front end of the upper mouthpiece body.

Further, the limiting boss is located at a rear end of the lower mouthpiece body, and the plurality of through holes on the limiting boss are distributed on the limiting boss in a front-rear direction.

Further, a cavity is arranged inside the limiting boss, a flexible boss is arranged at a top end of the lower dental pad, the flexible boss is located inside the cavity, and the flexible boss is provided with inserting slots corresponding to the through holes.

Further, positioning holes are formed on the top plate and the base plate, and positioning columns are arranged on the lower dental pad and the upper dental pad, respectively, where the positioning columns are located inside the positioning holes.

Further, an end of each of the positioning columns is provided with a fixing portion, and a diameter of the fixing portion is larger than a diameter of each of the positioning holes.

Further, supporting bosses are arranged at both ends of a rear end of the lower dental pad.

Further, protruding strips are arranged at a top end of the upper dental pad and a bottom end of the lower dental pad.

Further, flexible flanges are arranged on front sides of the upper dental pad and the lower dental pad, and gaps for accommodating anterior teeth are formed between the flexible flanges and the protruding strips.

Further, wavy edges are arranged on the sides of the first outer baffle and the second outer baffle facing tooth roots.

According to the technical solution of the present disclosure, the upper mouthpiece body and the lower mouthpiece body of the anti-snoring mouthpiece are made of the hard plastic, which enhances the stability of the mouthpiece and reduces the risk of deformation during use, thereby ensuring the durability of the anti-snoring effect. By arranging the limiting boss and the limiting insertion strip structure, the user can adjust the relative position between the upper mouthpiece and the lower mouthpiece according to actual needs. This design improves the versatility of the mouthpiece, and the mouthpiece can be flexibly adjusted according to structural features of individual oral cavities and respiratory tracts, which increases the comfort of wearing and enhances the anti-snoring effect. The airway protrusion is arranged at the bottom end of the base plate of the upper mouthpiece body, and when the airway protrusion abuts against the top plate of the lower mouthpiece body, an airway for airflow to pass through is formed. This design helps to keep the airflow smooth and reduce breathing resistance, so as to minimize the occurrence of snoring. The limiting boss of the lower mouthpiece body is provided with the through holes and is combined with the flexible boss on the lower dental pad to form a fixing structure. This design not only ensures the stability of the mouthpiece, but also allows a certain degree of airway adjustment to meet the needs of different patients.

Other features and advantages of the present disclosure will be set forth in the following specification, and will partially become apparent in the specification, or may be learned by implementing the present disclosure. The objective and other advantages of the present disclosure can be achieved and obtained by means of structures specially indicated in the written specification and accompanying drawings.

### BRIEF DESCRIPTION OF THE DRAWINGS

The present disclosure will be described in detail below with reference to accompanying drawings to further clarify the above advantages of the present disclosure.

FIG. 1 is a schematic diagram of an anti-snoring mouthpiece according to the present disclosure.

FIG. 2 is a schematic diagram of a front end of an anti-snoring mouthpiece according to the present disclosure.

FIG. 3 is a schematic diagram of breakdown of an anti-snoring mouthpiece according to the present disclosure.

FIG. 4 is an exposed view of an anti-snoring mouthpiece according to the present disclosure.

FIG. 5 is a first exploded view of an anti-snoring mouthpiece according to the present disclosure.

FIG. 6 is a second exploded view of an anti-snoring mouthpiece according to the present disclosure.

### DETAILED DESCRIPTIONS OF THE EMBODIMENTS

Embodiments of the present disclosure are described in detail below, and examples of the embodiments are shown in accompanying drawings, throughout which identical or similar reference numerals denote identical or similar elements or elements having identical or similar functions. The embodiments described with reference to the accompanying drawings are exemplary and only intended to explain the present disclosure, instead of being construed as limiting the present disclosure.

In the description of the present disclosure, it is to be understood that the terms “length”, “width”, “upper”, “lower”, “front”, “back”, “left”, “right”, “vertical”, “horizontal”, “top”, “bottom”, “inner”, “outer”, etc. indicate azimuthal or positional relations based on those shown in the drawings only for ease of description of the present disclosure and for simplicity of description, and are not intended to indicate or imply that the referenced device or element must have a particular orientation and be constructed and operative in a particular orientation, and thus may not be construed as a limitation on the present disclosure.

Furthermore, the terms “first” and “second” are merely for the purpose of description, and cannot be construed as indicating or implying relative importance or implicitly specifying the number of technical features indicated. Thus, a feature defined with “first” and “second” may explicitly or implicitly include one or more of the features. In the description of the present disclosure, “a plurality of” means two or more, unless expressly specified otherwise.

In the embodiments of the present disclosure, unless otherwise explicitly specified and defined, the terms “mounting”, “connecting”, “connection”, “fixing”, etc. should be understood Embodiment in a broad sense, for example, they may be a fixed connection, a detachable connection, or an integrated connection; may be a mechanical connection, or an electrical connection; may be a direct connection, or an indirect connection via an intermediate medium; and may be communication inside two elements, or an interactive relation between two elements. For those of ordinary skill in the art, the specific meanings of the above terms in the present disclosure may be understood according to specific circumstances.

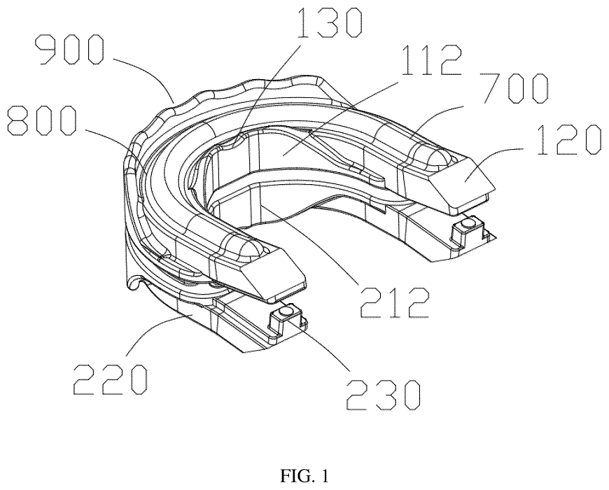

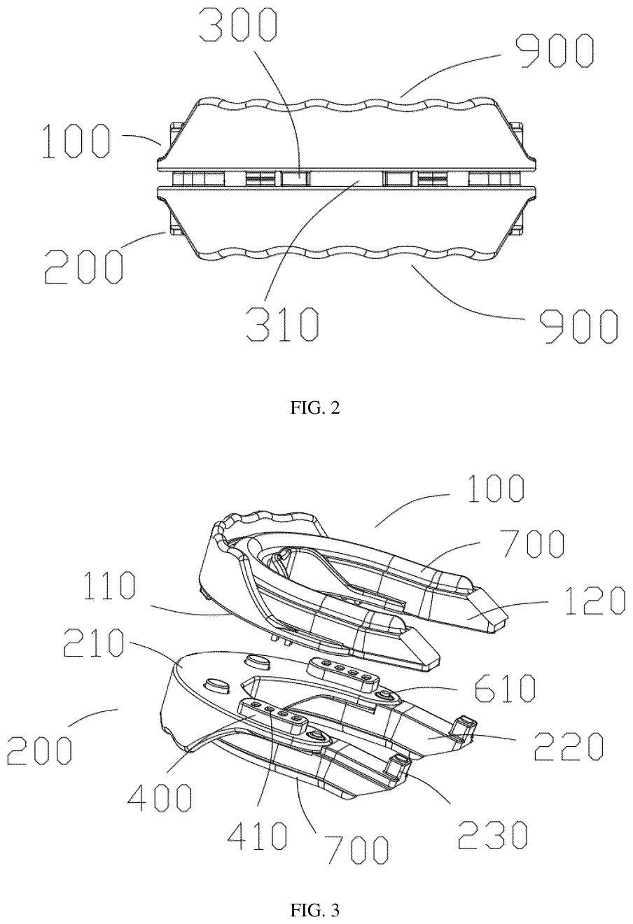

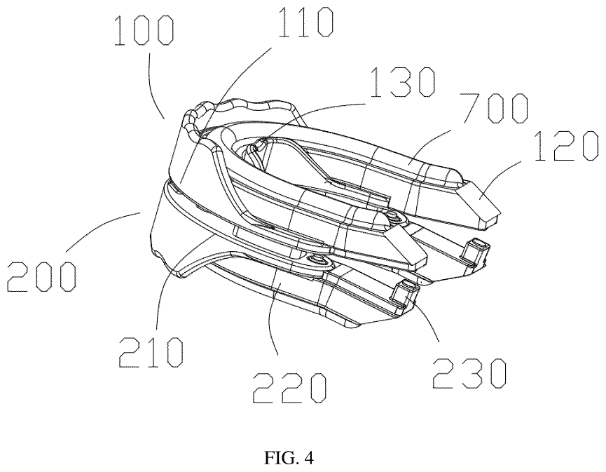

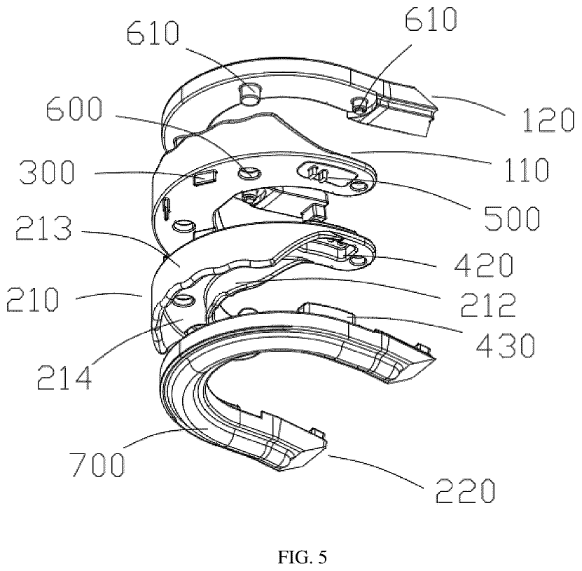

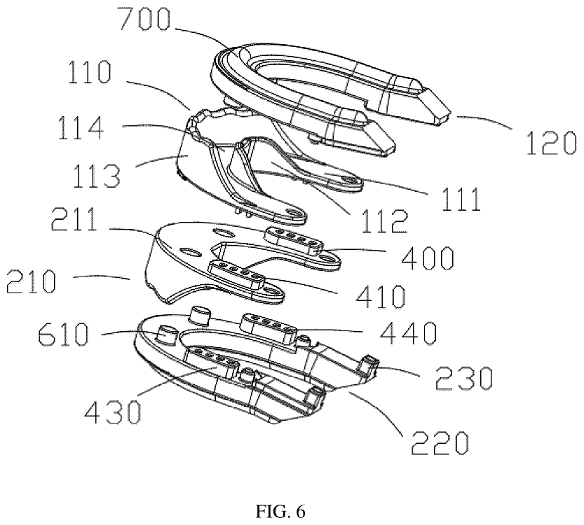

With reference to FIGS. 1-6, an anti-snoring mouthpiece is designed to overcome some common defects of anti-snoring devices in the prior art, such as deformation, insecure fixation, and incapability to adjust a relative position between an upper mouthpiece and a lower mouthpiece. By combining a hard plastic material and a flexible material, the present disclosure provides a stable and comfortable solution. The anti-snoring mouthpiece includes an upper mouthpiece 100 and a lower mouthpiece 200.

The upper mouthpiece 100 includes an upper mouthpiece body 110 and an upper dental pad 120, where the upper mouthpiece body 110 includes a base plate 111, a first inner baffle 112, and a first outer baffle 113. A first accommodating cavity 114 is formed by connecting the base plate 111, the first inner baffle 112 and the first outer baffle 113, and the upper dental pad 120 is embedded in the first accommodating cavity 114.

The lower mouthpiece 200 includes a lower mouthpiece body 210 and a lower dental pad 220, where the lower mouthpiece body 210 includes a top plate 211, a second inner baffle 212 and a second outer baffle 213. A second accommodating cavity 214 is formed by connecting the top plate 211, the second inner baffle 212 and the second outer baffle 213, and the lower dental pad 220 is embedded in the second accommodating cavity 214.

Either the upper mouthpiece 100 or the lower mouthpiece 200 includes a mouthpiece body and a dental pad, and the dental pad is embedded in an accommodating cavity of the mouthpiece body. For the purpose of wearing, a patient can fix the upper dental pad 120 and the lower dental pad 220 on teeth of an upper jaw and a lower jaw respectively.

The upper mouthpiece body 110 and the lower mouthpiece body 210 are made of hard plastic, and the upper dental pad 120 and the lower dental pad 220 are made of flexible rubber. The upper mouthpiece body 110 and the lower mouthpiece body 210 are made of the hard plastic to improve overall structural stability of the mouthpiece and prevent effect decline caused by deformation under pressure during use. The upper dental pad 120 and the lower dental pad 220 are made of the flexible rubber to ensure good fitting with the teeth and enhance wearing comfort.

Circumferences of the upper mouthpiece body 110 and the lower mouthpiece body 210 are smaller than those of the upper dental pad 120 and the lower dental pad 220 respectively, and the upper dental pad 120 and the lower dental pad 220 extend from both ends of the upper mouthpiece body 110 and the lower mouthpiece body 210 and extend backward respectively. The upper dental pad and the lower dental pad extend from both ends of the mouthpiece bodies and extend backward, meaning that the dental pads are larger than the mouthpiece bodies in area. This design allows the dental pads to cover a larger surface area of the teeth, thereby achieving better fitness. The dental pads made of the flexible material can be in better contact with the teeth and oral soft tissues, which reduces pressure on a wearer and improves a level of comfort. Relatively short ends of the mouthpiece bodies reduce the pressure of the hard plastic material on the oral cavity of the wearer. The mouthpiece bodies mainly play a role of supporting and fixing the dental pads, and are designed to be short to reduce the foreign body sensation in the oral cavity and avoid discomfort. Portions of the dental pads extending beyond the mouthpiece bodies can be made in contact with more teeth to enhance the fixing effect. Even when some pressure or friction occurs in the wearing process, the longer dental pads are not easily displaced, and the anti-snoring device maintains at a proper position, thereby ensuring a stable anti-snoring effect.

A bottom end of the base plate 111 of the upper mouthpiece body 110 is provided with an airway protrusion 300, and when the airway protrusion 300 abuts against a top end of the top plate 211 of the lower mouthpiece body 210, an airway 310 for airflow to pass through is formed between the upper mouthpiece body 110 and the lower mouthpiece body 210.

The top end of the top plate 211 of the lower mouthpiece body 210 is provided with a limiting boss 400, a plurality of through holes 410 are formed in the limiting boss 400, the bottom end of the base plate 111 of the upper mouthpiece body 110 is provided with a limiting insertion strip 500, the limiting insertion strip 500 is located in the through hole 410 to fix the upper mouthpiece 100 and the lower mouthpiece 200, and the through hole 410 corresponding to the limiting insertion strip 500 is adjusted to adjust a relative position between the upper mouthpiece 100 and the lower mouthpiece 200.

The airway protrusion 300 is arranged at the bottom end of the base plate 111 of the upper mouthpiece body 110 and matches the top plate 211 of the lower mouthpiece body 210 to form an airway 310 to ensure smooth airflow in the wearing process. The design of the limiting boss 400 and the limiting insertion strip allows for the adjustability of the relative position between the upper mouthpiece and the lower mouthpiece, to meet the needs of different patients. The hard plastic mouthpiece bodies support the mouthpiece to keep the shape during use, and the dental pads made of the flexible rubber offer a comfortable fitness experience. The bottom end of the base plate 111 of the upper mouthpiece body 110 is provided with the airway protrusion 300, and when the upper mouthpiece and the lower mouthpiece are worn, the airway protrusion 300 is in contact with the top plate 211 of the lower mouthpiece body 210, which forms the airway 310 for airflow to pass through. The design of the airway 310 helps to keep the airflow smooth and reduce breathing resistance, so as to minimize the occurrence of snoring. The top plate 211 of the lower mouthpiece body 210 is provided with the limiting boss 400, and the base plate 111 of the upper mouthpiece body 110 is provided with the limiting insertion strip 500. The limiting insertion strip 500, through the through hole 410 on the limiting boss 400, fixes the relative position between the upper mouthpiece 100 and the lower mouthpiece 200. By selecting different through holes 410, a user can adjust the relative position between the upper mouthpiece and the lower mouthpiece according to personal needs of comfort and effect.

Through the structural design of the upper mouthpiece and the lower mouthpiece, the anti-snoring mouthpiece enables the wearer's lower jaw to move forward when wearing. Specifically, when the user puts the upper mouthpiece and the lower mouthpiece on the upper and lower teeth, a structure of the limiting insertion strip 500 and the through hole 410 between the upper mouthpiece body 110 and the lower mouthpiece body 210 allows for adjusting the relative position between the upper mouthpiece and the lower mouthpiece, such that a position of the lower jaw can be adjusted. Forward movement of the lower jaw can increase space in a throat area, reduce backward movement of soft tissues such as the soft palate and the tongue root, and prevent them from obstructing the airway 310 during sleep. The airway 310 is kept open to reduce the occurrence of airflow obstruction, so as to reduce or eliminate the occurrence of snoring. The airway protrusion 300 is designed to be located at the bottom end of the base plate 111 of the upper mouthpiece body 110, and when the upper mouthpiece 100 is in contact with the lower mouthpiece 200, the airway protrusion 300 matches the top end of the top plate 211 of the lower mouthpiece body 210 to form a passage for airflow. The design of the airway 310 ensures that even when the oral cavity is closed, there is an open airflow passage that facilitates smooth flow of air through the oral cavity and the throat, thereby reducing the occurrence of snoring. The existence of the airway 310 compensates for the potential closure of the oral cavity when the lower jaw moves forward, thereby further ensuring smooth breathing through a respiratory tract. Through a combination of the mouthpiece bodies made of the hard plastic and the dental pads made of the flexible rubber, the anti-snoring mouthpiece is capable of not only securely moving the lower jaw forward but also applying moderate tension to the soft tissues inside the oral cavity. The tension can effectively prevent the tongue and the soft palate from moving backward or collapsing during sleep, thereby maintaining the openness of the respiratory tract. This design helps to reduce the obstruction of the airway 310 during sleep, thereby effectively reducing the phenomena of snoring and related apnea.

In this embodiment, a notch 130 is formed in a middle of the first inner baffle 112 of the upper mouthpiece body 110. In the design of the anti-snoring mouthpiece, consideration is given to the diversity of oral anatomical structures and individual differences, particularly the potential discomfort or pain that could arise from pressure of the mouthpiece on a position of a labial frenulum. Therefore, in this embodiment, the notch 130 is formed in the middle of the first inner baffle 112 of the upper mouthpiece body 110 to avoid contact with the labial frenulum. This design not only improves the comfort of wearing, but also avoids discomfort of the wearer or displacement of the mouthpiece caused by the pressure on the labial frenulum.

Before using the anti-snoring mouthpiece, the user should ensure his/her oral hygiene. The mouthpiece is designed and customized based on an oral cavity structure of the user, to ensure the notch 130 is formed in the middle of the inner baffle at the position of the labial frenulum. The upper mouthpiece 100 and the lower mouthpiece 200 are placed on the teeth of the upper jaw and the lower jaw respectively. Because the notch 130 is formed in the middle of the first inner baffle 112 of the upper mouthpiece body 110, the position of the notch 130 is naturally not in contact with the labial frenulum, thereby ensuring no pressure on the labial frenulum. The positions of the upper mouthpiece and the lower mouthpiece are adjusted to move the lower jaw forward moderately, so as to open the respiratory tract and maintain smooth airflow through the airway protrusion 300. After waking up, the user needs to gently remove the upper mouthpiece and the lower mouthpiece for cleaning and maintaining them. The presence of the notch 130 ensures no additional friction or harm to the labial frenulum.

Due to the special physiological position of the labial frenulum, arrangement of the notch 130 in the middle of the inner baffle avoids pressure of the mouthpiece on the labial frenulum. This design greatly improves the comfort of wearing, allowing the user to use the mouthpiece without disturbance during sleep. Avoiding direct pressure on the labial frenulum reduces the discomfort or pain that may arise from wearing the mouthpiece, and allows the user to use more comfortably for a long period.

In this embodiment, the airway protrusion 300 is located at an outer edge of a front end of the upper mouthpiece body 110. A structure of the airway protrusion 300 at the outer edge of the front end of the upper mouthpiece body 110 ensures that even when the lower mouthpiece 200 is displaced forward during use, the airway 310 remains unobstructed. Forward movement of the lower jaw is a common approach used by many anti-snoring devices, and the forward movement can increase the space in the respiratory tract and reduce the obstruction of the airway 310. The airway protrusion 300 designed is located at the outer edge of the upper mouthpiece body 110, which maintains contact with the lower mouthpiece 200 even when it is displaced, thereby ensuring continued openness of the airway 310.

The user wears the upper mouthpiece and the lower mouthpiece on the teeth of the upper jaw and the lower jaw, respectively. The airway protrusion 300 is designed to be located at the outer edge of the front end of the upper mouthpiece body 110 to facilitate contact with the lower mouthpiece 200 during wearing. During wearing, the lower mouthpiece 200 can be fine-tuned forward according to the user's needs. This adjustment results in the forward movement of the lower jaw, thereby increasing the space in the airway 310 and reducing the occurrence of snoring. During the forward movement of the lower jaw, the airway protrusion 300 at the outer edge of the front end of the upper mouthpiece body 110 continues contact with the lower mouthpiece 200, and even when the lower mouthpiece 200 is displaced forward, the airway protrusion 300 is capable of ensuring that the airway 310 formed between the upper mouthpiece and the lower mouthpiece remains unobstructed. When the user enters a sleep state after wearing the mouthpiece, the mouthpiece will automatically adjust the position of the lower jaw and maintain the openness of the airway 310. This design ensures that even when displacement occurs at night, the airway 310 remains unobstructed.

The airway protrusion 300 located at the outer edge of the front end of the upper mouthpiece body 110 is capable of effectively avoiding the displacement of the lower mouthpiece 200, and ensuring the continuity and smoothness of the airway 310, thereby preventing the obstruction of the airway 310 due to the movement of the lower jaw. This design allows the lower mouthpiece 200 to move forward during use, which meets the need for the natural forward movement of the lower jaw while ensuring the effectiveness of the anti-snoring device.

In this embodiment, the limiting boss 400 is located at a rear end of the lower mouthpiece body 210, and the plurality of through holes 410 on the limiting boss 400 are distributed on the limiting boss 400 in a front-rear direction. An area of the rear end of the lower mouthpiece body 210 is usually spacious, and is capable of accommodating the limiting boss 400 and the plurality of through holes 410. When the limiting boss 400 is arranged at a front end of the lower mouthpiece body 210, the limited space at the front end thereof may restrict the range and gears of adjustment. The rear end of the lower mouthpiece body 210 is usually close to a tail end of a dental arch, thereby achieving better support and stability. Through arrangement of the limiting boss 400 at the rear end of the lower mouthpiece body 210, the relative position between the upper mouthpiece body 110 and the lower mouthpiece body 210 can be fixed more stably, thereby reducing the risk of displacement. The limiting boss 400 is arranged at the rear end of the lower mouthpiece body 210, and the plurality of through holes 410 can be arranged in long rear space of the mouthpiece, such that a wider range of front-rear adjustment is provided. This design can meet the personalized needs of different users, and ensure that the adjustment over the forward movement of the lower jaw can be achieved within a larger range. When the limiting boss 400 is arranged at the front end of the lower mouthpiece body 210, adjustment gears might be reduced due to limited space, thereby affecting applicability of the mouthpiece and the comfort of the user. This design aims to meet the needs of front-rear adjustment between the upper mouthpiece and the lower mouthpiece. Further, U-shaped distribution of the mouthpiece combined in this design, makes a rear position of the limiting boss 400 become a most suitable adjustment point. Further, the limiting boss 400 at the rear end and the airway protrusion 300 at the front end match each other, which effectively ensures smoothness of a breathing passage inside the oral cavity, and avoids complete closure of the mouth during sleep, thereby maintaining smooth oral breathing.

By adjusting the position of the through hole 410 on the limiting boss 400, the user can select an appropriate front-rear position to adjust the relative position between the upper mouthpiece and the lower mouthpiece. This process allows the lower jaw to move forward moderately, such that the respiratory tract is opened. Through the combination of the limiting boss 400 and the airway protrusion 300 at the front end, even when the oral cavity is closed during sleep after the user wears the mouthpiece, the airway protrusion 300 is capable of effectively maintaining the openness of the breathing passage inside the oral cavity and avoiding breathing difficulties. This design is particularly suitable for users who are accustomed to oral breathing, and is capable of effectively preventing snoring or apnea caused by obstruction of the respiratory tract.

In this embodiment, a cavity 420 is arranged inside the limiting boss 400, a flexible boss 430 is arranged at a top end of the lower dental pad 220, the flexible boss 430 is located inside the cavity 420, and the flexible boss 430 is provided with inserting slots 440 corresponding to the through holes 410. The user wears the upper mouthpiece and the lower mouthpiece on the teeth of the upper jaw and the lower jaw, respectively. The flexible boss 430 at the top end of the lower dental pad 220 is inserted into the cavity 420 inside the limiting boss 400 to ensure proper positioning. The user can adjust the relative position between the upper mouthpiece and the lower mouthpiece by inserting the limiting insertion strip into the corresponding inserting slot 440 on the flexible boss 430. A combination of the limiting insertion strip and the inserting slots 440 fixes the positions of the upper mouthpiece body and the lower mouthpiece body while maintaining the stability of the entire structure of the mouthpiece.

The user inserts the limiting insertion strip into the corresponding inserting slot 440 on the flexible boss 430 as needed, and the inserting slots 440 and the through holes 410 on the limiting boss 400 match each other to ensure the fixation and adjustment of the upper mouthpiece and the lower mouthpiece. Due to the design of the flexible boss 430, the limiting insertion strip is not in direct contact with the teeth during fixation, thereby effectively avoiding the pressure and friction of the limiting insertion strip on the teeth, and improving the comfort of wearing. When necessary, the user can change the relative position between the upper mouthpiece and the lower mouthpiece by adjusting the position of the limiting insertion strip, and the flexible boss 430 ensures that the limiting insertion strip remains stable and does not interfere with the teeth during adjustment. By arranging the cavity 420 inside the limiting boss 400 and combining the design of the flexible boss 430 at the top end of the lower dental pad 220, the present disclosure successfully achieves the stability of the limiting insertion strip and the avoidance of interference with the tooth. The flexible boss 430 not only ensures the stability of the limiting insertion strip during use but also reduces the interference with the teeth and the discomfort of the user due to use of the flexible material. This design enhances the user experience of the anti-snoring mouthpiece, better meets the needs of different users, and improves the overall comfort and effect.

In this embodiment, positioning holes 600 are formed on the top plate 211 and the base plate 111, and positioning columns 610 are arranged on the lower dental pad 220 and the upper dental pad 120, respectively, where the positioning columns 610 are located inside the positioning holes 600. The positioning holes 600 are formed on the top plate 211 and base plate 111 of the anti-snoring mouthpiece, and corresponding positioning columns 610 are arranged on the lower dental pad 220 and the upper dental pad 120 respectively, where the positioning columns 610 are fixed on the mouthpiece bodies through the positioning holes 600. This design aims to improve the efficiency of production and assembly, and particularly under the conditions of silicone injection molding, the combination of the positioning holes 600 and the positioning columns 610 can significantly improve the stability of combining the dental pads and the mouthpiece bodies.

During production, the dental pads are fixed on the mouthpiece bodies through the silicone injection molding process. In the injection molding process, the positioning columns 610 will be accurately inserted into the positioning holes 600 on the top plate 211 and the base plate 111. This design improves the production efficiency and ensures a firm combination between the dental pads and the mouthpiece bodies. When the user wears the mouthpiece, due to the fixing effect of the positioning holes 600 and the positioning columns 610, the positions of the dental pads in the mouthpiece bodies will not easily change, thus ensuring the overall stability and effect of the mouthpieces. Due to the stable combination of the positioning columns 610 and the positioning holes 600, the dental pads and the mouthpiece bodies are connected very firmly and are not prone to loosening during daily use. The user only needs to perform regular cleaning and maintenance to achieve the long-term use of the mouthpieces.

In this embodiment, an end of each of the positioning columns 610 is provided with a fixing portion, and a diameter of the fixing portion is larger than a diameter of each of the positioning holes 600. A core objective of this design is to enhance the strength of combining between the dental pads and the mouthpiece bodies in a manner of mechanical locking, which avoids detachment of the dental pads during use. In the production process, the dental pads are combined with the mouthpiece bodies through the positioning columns 610. The end of each of the positioning columns 610 is provided with the fixing portion, the fixing portion needs to pass through the corresponding positioning hole 600 during assembly, and since its diameter is larger than that of each of the positioning holes 600, a mechanical locking effect will be formed after the fixing portion passes through the corresponding positioning hole 600. This locking mechanism ensures that the dental pads are firmly fixed on the mouthpiece bodies after the injection molding or assembly and are not prone to loosening or detachment. When the user wears the mouthpiece, the fixing portion of each of the positioning columns 610 is capable of ensuring that the dental pads remain stable during use and will not fall off due to oral movement or any other external force. Due to the existence of the fixing portion, the dental pads and the mouthpiece bodies are connected very firmly. The user does not need to worry about the loosening or displacement of the dental pads during daily use and only needs to perform the regular cleaning and maintenance. The design of the fixing portion greatly reduces the risk of detachment of the dental pads during use, and ensures the continued effect of the mouthpiece, thereby making the user feel more at ease during wearing.

In this embodiment, supporting bosses 230 are arranged at both ends of a rear end of the lower dental pad 220. The main problem to be solved is lack of support for a portion of the upper dental pad 120 extending out due to the forward displacement of the lower mouthpiece 200 and a shorter length of the mouthpiece body compared to that of the dental pad when the mouthpiece is worn. The supporting bosses 230 not only provide necessary support for both ends of the upper dental pad 120, but also reduce the foreign body sensation and improve the comfort of wearing due to use of the flexible material.

The lower mouthpiece 200 may move forward when worn, and such displacement causes the extended portion of the upper dental pad 120 to lose support. The supporting bosses 230 are located at the rear end of the lower dental pad 220, and the extended portion of the upper dental pad 120 is supported to ensure stable contact between the upper dental pad and the lower dental pad in the case of occlusive contact, particularly providing more effective support for posterior molars. The supporting bosses 230 support both ends of the upper dental pad 120, causing more even occlusion, and particularly providing additional support for the posterior molars, thereby ensuring more stable and reliable contact between the upper dental pad and the lower dental pad. The use of a flexible support material at a rear end of the oral cavity reduces the foreign body sensation caused by a hard material while providing support. The flexible support material is capable of adapting to dynamic changes inside the oral cavity, and providing a comfortable wearing experience.

In this embodiment, protruding strips 700 are arranged at a top end of the upper dental pad 120 and a bottom end of the lower dental pad 220. These protruding strips 700 are designed to enhance the stability in the case of molar occlusion. Through the protruding strips 700 added on the dental pads, the molars can be tight contact with the protruding strips 700 in the case of occlusion, which forms a large friction force, thereby achieving a better fixing effect. This design effectively reduces the displacement between the teeth and the dental pads, thereby ensuring that the mouthpiece remain stable during wearing.

The protruding strips 700 are designed to adapt to different oral cavity structures and tooth shapes and achieve consistent stability, such that the mouthpiece are applicable to a wider range of users. The molars occlude on the protruding strips 700 of the upper dental pad 120 and the lower dental pad 220, which forms a stable friction force. This friction force effectively fixes the positions of the teeth and the dental pads, and prevents sliding or displacement during wearing. Due to the design of the protruding strips 700 during use, the teeth can be firmly fixed on the dental pads, thereby reducing displacement caused by the oral movement or any other external force, and ensuring the continued effect of the mouthpiece.

In this embodiment, flexible flanges are arranged on front sides of the upper dental pad 120 and the lower dental pad 220, and gaps 800 for accommodating anterior teeth are formed between the flexible flanges and the protruding strips 700. The anterior teeth are mainly responsible for cutting and biting food, are flat and shovel-like, and are located in a front portion of the oral cavity. The molars are mainly responsible for grinding and chewing food, and are wide and large with occlusal cusps, and are located in a rear portion of the oral cavity.

To solve the problem that the anterior teeth have a relatively small occlusal surface area and easily slip on the protruding strips 700, the flexible flanges are designed on the front sides of the upper dental pad 120 and the lower dental pad 220. The gaps 800 for accommodating anterior teeth are formed between the flexible flanges and the protruding strips 700. An objective of this design is to provide a stable positioning space for the anterior teeth through the combination of the flexible flanges and the protruding strips 700, so as to prevent the anterior teeth from slipping during use and reduce hard collision between the anterior teeth and the mouthpiece bodies, thereby improving the comfort and stability of wearing.

A dedicated gap 800 is provided for the anterior teeth, which prevents the slipping of the anterior teeth on the protruding strips 700, and ensures the overall stability of the mouthpiece.

In this embodiment, wavy edges 900 are arranged on the sides of the first outer baffle 113 and the second outer baffle 213 facing tooth roots. The design of the wavy edges 900 enables to better adapt to natural shapes of gums and tooth roots, avoiding the pressure of straight or sharp edges on the tooth roots and gums. This design is capable of reducing the stimulation of the mouthpiece on soft tissues, and preventing discomfort or pain caused during wearing. Through the design of the wavy edges 900, the mouthpiece can fit the gums and tooth roots more naturally, which avoids discomfort caused by pressure, and effectively improves comfort of the user particularly when wearing the mouthpiece for a long time. The design of the wavy edges 900 enables to better adapt to the oral cavity structures of different users, particularly natural undulations of gum lines. This design helps the mouthpiece to be fixed on the teeth more firmly, and reduces problems of adaptability caused by differences in oral cavity structures. The wavy edges 900 are gentler than straight edges, which achieves a certain cushioning effect when the mouthpiece are in contact with the gums. This design enables to reduce the friction and pressure between the mouthpiece and the gums, thereby improving the comfort of wearing.

Finally, it should be noted that the above is only a preferred embodiment of the present disclosure, and is not intended to limit the present disclosure. Although the present disclosure has been described in detail with reference to the above embodiment, for those skilled in the art, it is still apparent that the technical solutions described in the above embodiment may be modified, or some technical features thereof may be equivalently replaced. Any modifications, equivalent substitutions, improvements, etc. within the spirit and principles of the present disclosure are intended to fall within the scope of protection of the present disclosure.

What is claimed is:

1. An anti-snoring mouthpiece, comprising an upper mouthpiece and a lower mouthpiece;
the upper mouthpiece comprises an upper mouthpiece body and an upper dental pad, wherein the upper mouthpiece body comprises a base plate, a first inner baffle, and a first outer baffle; a first accommodating cavity is formed by connecting the base plate, the first inner baffle and the first outer baffle, and the upper dental pad is embedded in the first accommodating cavity;
the lower mouthpiece comprises a lower mouthpiece body and a lower dental pad, wherein the lower mouthpiece body comprises a top plate, a second inner baffle and a second outer baffle; a second accommodating cavity is formed by connecting the top plate, the second inner baffle and the second outer baffle, and the lower dental pad is embedded in the second accommodating cavity;
the upper mouthpiece body and the lower mouthpiece body are made of hard plastic, and the upper dental pad and the lower dental pad are made of flexible rubber; circumferences of the upper mouthpiece body and the lower mouthpiece body are smaller than those of the upper dental pad and the lower dental pad respectively, and the upper dental pad and the lower dental pad extend from both ends of the upper mouthpiece body and the lower mouthpiece body and extend backward respectively; wherein the upper dental pad and the lower dental pad are configured to extend beyond the respective upper and lower mouthpiece bodies to provide posterior dental support while reducing soft tissue compression in the oral cavity;
a bottom end of the base plate of the upper mouthpiece body is provided with an airway protrusion, and when the airway protrusion abuts against a top end of the top plate of the lower mouthpiece body, an airway for airflow to pass through is formed between the upper mouthpiece body and the lower mouthpiece body; and
the top end of the top plate of the lower mouthpiece body is provided with a limiting boss, wherein a plurality of through holes are formed in the limiting boss, the bottom end of the base plate of the upper mouthpiece body is provided with a limiting insertion strip, which is configured to be inserted into a selected one of the plurality of through holes to fix the upper mouthpiece to the lower mouthpiece, adjusting the selection of the through hole corresponding to the limiting insertion strip allows adjustment of the relative position between the upper mouthpiece and the lower mouthpiece.

2. The anti-snoring mouthpiece according to claim 1, wherein a notch is formed in a middle of the first inner baffle of the upper mouthpiece body.

3. The anti-snoring mouthpiece according to claim 1, wherein the airway protrusion is located at an outer edge of a front end of the upper mouthpiece body.

4. The anti-snoring mouthpiece according to claim 1, wherein the limiting boss is located at a rear end of the lower mouthpiece body, and the plurality of through holes on the limiting boss are distributed on the limiting boss in a front-rear direction.

5. The anti-snoring mouthpiece according to claim 4, wherein a cavity is arranged inside the limiting boss, a flexible boss is arranged at a top end of the lower dental pad, the flexible boss is located inside the cavity, and the flexible boss is provided with inserting slots corresponding to the plurality of through holes.

6. The anti-snoring mouthpiece according to claim 1, wherein positioning holes are formed on the top plate and the base plate, and positioning columns are arranged on the lower dental pad and the upper dental pad, respectively, wherein the positioning columns are located inside the positioning holes.

7. The anti-snoring mouthpiece according to claim 6, wherein an end of each of the positioning columns is provided with a fixing portion, and a diameter of the fixing portion is larger than a diameter of each of the positioning holes.

8. The anti-snoring mouthpiece according to claim 1, wherein supporting bosses are arranged at both ends of a rear end of the lower dental pad.

9. The anti-snoring mouthpiece according to claim 1, wherein protruding strips are arranged at a top end of the upper dental pad and a bottom end of the lower dental pad.

10. (canceled)

11. (canceled)
